(19) United States
(12) Patent Application Publication
(10) Pub. No.: US 2016/0256064 A1
(43) Pub. Date: Sep. 8, 2016

(54) METHOD AND DEVICE TO PREDICT ADVERSE CARDIOVASCULAR EVENTS AND MORTALITY FROM AN ELECTROCARDIOGRAM-BASED VALIDATED RISK SCORE

(71) Applicant: Edward Harvey Estes, JR., Durham, NC (US)

(72) Inventor: Edward Harvey Estes, JR., Durham, NC (US)

(21) Appl. No.: 15/057,851

(22) Filed: Mar. 1, 2016

Related U.S. Application Data
(60) Provisional application No. 62/126,739, filed on Mar. 2, 2015.
(60) Provisional application No. 62/181,590, filed on Jun. 18, 2015.

(51) Int. Cl. A61B 5/04

(52) U.S. Cl. CPC A61B 5/04012

(57) ABSTRACT

The present invention is directed to methods for predicting risk of adverse cardiovascular events and/or mortality in an individual comprises the steps of: a) recording an electrocardiogram from the individual; b) analyzing the electrocardiogram to detect the presence of wave form elements; c) calculating a risk score based on the presence of the wave form elements; and d) predicting risk of adverse cardiovascular events and/or mortality based on the risk score. In another aspect, calculation of the risk score is based on a longitudinal assessment of wave form elements and adverse cardiovascular events and mortality in a population-based cohort study. In yet another aspect, steps (a) to (c) are performed by a compatible recording instrument programmed to detect, quantitate, and analyze the wave form elements and calculate the risk score based upon the wave form elements.

### CROSS-REFERENCE TO RELATED APPLICATIONS

This application claims the benefit of U.S. Provisional Application No. 62/126,739, filed Mar. 2, 2015, and U.S. Provisional Application No. 62/181,590, filed Jun. 18, 2015, the entire contents of which are incorporated by reference herein in their entireties.

### FEDERALLY SPONSORED RESEARCH OR DEVELOPMENT

This invention was based, in part, on data from the Atherosclerosis in Communities Study under Federal Contract Nos. HHSN268201100005C, HHSN268201100006C, HHSN268201100007C, HHSN268201100008C, HHSN268201100009C, HHSN268201100010C, HHSN268201100011C, and HHSN268201100012C awarded by the National Heart, Lung, and Blood Institute. Accordingly, the Federal Government has certain rights to this invention.

### BACKGROUND

At the present time there are no easy methods for obtaining an objective, scientifically validated measure of the future risk of new events or death due to cardiovascular events, such as heart attacks, heart failure, stroke, and sudden arrhythmias. For the past half century, the electrocardiogram (ECG) has been used primarily to indicate the presence of enlargement of the heart. But, this process has been plagued by a lack of sensitivity—many patients with enlargement have no positive ECG indicators. More recently, new imaging technologies such as ultrasound and magnetic resonance imaging now provide a more direct and more accurate method for detecting increased heart size, though they are considerably more expensive. Attempts to make the ECG more sensitive have always resulted in an unacceptable level of false positives. The US Preventive Services Task Force has advised against use of the ECG as an indicator of impending coronary heart disease because of this absence of scientifically valid evidence of usefulness.

Alternatives to the ECG as a predictive tool exist, but each of these alternatives has its own limitations. For example, there are many analyses of blood or serum for various components such as cholesterol, blood lipids, biomarkers for inflammation, and similar elements which are known to be in patients predisposed to cardiovascular disease. However, none have been demonstrated to have the required sensitivity and specificity to be useful as an overall predictor of future adverse events. In addition, most of these methods require obtaining of blood specimens by venipuncture, transport to a laboratory, and a later analysis, making the result unavailable until a later date.

In light of these issues, it is not surprising that companies formed for the purpose of providing a risk assessment tool using any of the methods described above have not produced a marketable test. For example, several companies have formed for the purpose of providing risk assessment on the basis of blood analyses for enzymes, biomarkers or unique proteins in the blood sample. None are known to be marketed at this time. One company proposes to use physiologic signals derived from the human body for screening purposes, including the ECG, but the signals would be processed by a complex mathematical process which has yet to be tested on a population. This method is not yet offered in the marketplace (1).

Accordingly, there is a need for an inexpensive, easily obtained, objective, and scientifically validated measure that predicts the future risk of new events or death due to cardiovascular events and/or related biomarker features.

### BRIEF SUMMARY OF THE INVENTION

The present invention is directed to methods and devices for predicting risk of adverse cardiovascular events and/or mortality in an individual. In one aspect, the method for predicting risk of adverse cardiovascular events and/or mortality in an individual comprises the steps of: a) recording an electrocardiogram from the individual; b) analyzing the electrocardiogram to detect the presence of wave form elements; c) calculating a risk score based on the presence of the wave form elements; and d) predicting risk of adverse cardiovascular events and/or mortality based on the risk score.

In another aspect, calculation of the risk score is based on a longitudinal assessment of wave form elements and adverse cardiovascular events and mortality in a population-based cohort study. In a further aspect, the longitudinal assessment of wave form elements and adverse cardiovascular events and mortality in a population-based cohort study comprises analysis of the pattern of occurrence, intensity, and statistical response of wave form elements to predict specific adverse cardiovascular events and mortality.

In another aspect, the pattern of occurrence, intensity, and statistical response of wave form elements are also used to predict the presence of one or more genetic abnormalities in the individual. In another aspect, the pattern of occurrence, intensity, and statistical response of wave form elements are also used to predict the presence or absence of one or more biomarkers in the individual.

In a further aspect, the risk score is used to measure the efficacy of a medication, a surgical procedure, a dietary regime, or a treatment program in the individual, wherein an increase in the risk score is indicative of poor efficacy and a reduction in the risk score is indicative of positive efficacy.

In another aspect, total all-cause risk of mortality is predicted for an individual based on all identified wave form elements, and wherein each wave form element is rated and entered into the total risk score. In a further aspect, the total risk score is used to measure the efficacy of a medication, a surgical procedure, a dietary regime, or a treatment program in the individual, wherein an increase in the total risk score is indicative of poor efficacy and a reduction in the total risk score is indicative of positive efficacy.

In yet another aspect, steps (a) to (c) are performed by a compatible recording instrument programmed to detect, quantitate, and analyze the wave form elements and calculate the risk score based upon the wave form elements. In a further aspect, the compatible recording instrument is selected from the group consisting of an ECG recorder and analyzer, a computer, and a portable device such as a hand-held mobile device.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale.

FIG. 1 depicts Kaplan Meier Survival curves by levels of Romhilt-Estes (R-E) Score.

FIG. 2 depicts the ability of each of the six components of the R-E Score in detecting three cardiovascular outcomes: heart failure, coronary heart disease, and stroke. The response of each ECG component to three levels of correction are shown. The first level corrects for demographic factors (age, sex, race). The second level corrects for these plus CV risk factors. The third corrects for these two plus the other components of the R-E Score. The color of the bar indicates the p value of the hazard ratio (see key).

### DETAILED DESCRIPTION OF THE INVENTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Methods for Predicting Risk of Adverse Cardiovascular Events and/or Mortality

At the present time there are no easy methods for obtaining an objective, scientifically validated measure of the future risk of new events or death due to cardiovascular events, such as heart attacks, heart failure, stroke, and sudden arrhythmias. There are many analyses of blood or serum for various components such as cholesterol, blood lipids, biomarkers for inflammation, and similar elements which are known to be in patients predisposed to cardiovascular disease, but none have been demonstrated to have the sensitivity and specificity required to be useful as an overall predictor of future adverse complications. In spite of the fact that the ECG has been used for the past half century for the detection of enlargement of the heart, most often the left ventricle (left ventricular hypertrophy or LVH), the U.S. Preventive Services Task Force has advised against use of the ECG as an indicator of impending coronary heart disease because of this absence of scientifically valid evidence of usefulness.

In 1968, the inventor published a paper describing a new method for detecting left ventricular hypertrophy (LVH) using a point score system based on the occurrence of six abnormalities within the wave forms of the ECG (2). The presence of each of these abnormalities was scored as a specific number of points, and the total points for that ECG was used to predict LVH. This point score system, known as the Romhilt-Estes LVH score (R-E score), was unique in that it used all waves in the ECG instead of the ventricular depolarization component, the QRS complex, as was used in most other methods. It is still widely used for this purpose.

A major limitation of currently available technology, which includes the use of the ECG as a detector of LVH, and the use of various blood tests as a detector of conditions known to predispose to the development of heart disease, is that there have been no studies validating their sensitivity and effectiveness in predicting adverse cardiovascular events in a representative population over a span of years. Recently it has been shown that some indicators of left ventricular enlargement also have a correlation with adverse cardiovascular events, and that this is independent of the presence of increased cardiac mass (3). This has led some observers to conclude that the presence of increased mass and the presence of the ECG manifestations once attributed to increased LV mass are caused by separate but somehow related phenomena (4).

To further clarify these interrelationships, and to study the predictive characteristics of the R-E score and its component parts, the inventor and colleagues conducted a study in which the relationships between the presence of the six ECG phenomena used in the R-E score and all-cause mortality was studied in a population of 14,900 men and women who volunteered for the Arteriosclerosis Risk in Communities study, a National Institutes of Health sponsored epidemiological study of over 15,000 volunteers, which began in 1987, and in which the panel of initial and follow-up tests included an electrocardiogram (8). The original R-E score proved to be strongly correlated with all-cause mortality. An increase in the R-E score between the first and the first follow-up exam was even more strongly correlated. Four of the six ECG components that comprise the R-E score were also found to be strongly correlated with all-cause mortality, and each component was found to be independent of the others. These findings were judged to be of sufficient strength and consistency to serve as a guide to the physician in his/her care of individual patients. The totality of these observations serve as the major science base for this invention (5).

The six ECG components of the R-E score are: 1) increased amplitude of the R or S wave of the QRS complex in certain leads, 2) increase in the terminal negative portion of the P wave in lead V1, 3) deviation of the ST and T components in a direction opposite to the direction of the QRS in V5 or V6, in the absence of digitalis, 4) left axis deviation equal to or greater than −30 degrees, 5) QRS duration equal to or greater than 0.09 milliseconds, and 6) duration of QRS from onset to peak of R wave in V5 or V6 (“intrinsicoid deflection”) equal to or greater than 0.05 milliseconds. Left axis deviation and QRS duration (#s 4 and 5) did not prove to have independent predictive ability, but the other 4 were predictive at a P value of <0.0001 for all-cause mortality, after correction for age, sex, race, and demographic and clinical variables predisposing to heart disease. The two ECG components that failed to have independent predictive value for mortality were found in a later study to predict individual cardiovascular diseases, so they are still included in the calculation of the total risk score. These six ECG variables, with the threshold value of each recalibrated for optimal risk predictive ability, are utilized to generate the cardiovascular risk score on the ECG report for that individual. The relationships between the score and all-cause mortality have also been found to be present with cardiovascular mortality and the new incidence of cardiovascular disease.

In its main aspect, the invention is a method for using certain components of the electrocardiogram recorded from an individual to predict that individual's risk of adverse cardiovascular events, such as heart attacks, arrhythmias, congestive heart failure, strokes, and death. This information is obtained from an ECG, recorded on an instrument with the internal capability of measuring intervals, magnitudes, and polarity of waveforms, performing certain diagnostic analyses, and producing a written report for the responsible physician, in the form of a risk score. Such recording instruments are now considered as state of the art machines, and are made by several companies in the US and Europe, and available in most hospital and physician offices. These would need modification by their manufacturer to perform certain specific functions described below, and not included in the current analysis and reports. The invention consists of both the method of analysis and the compatible recording instrument, programmed to perform added analyses and generate added report content.

There is no existing technology today for the validated risk assessment function achieved by the new invention. As described above, for the past half century, the ECG has been used to indicate the presence of enlargement of the heart. This process has been plagued by a lack of sensitivity—many patients with enlargement have no positive ECG indicators. Attempts to improve this deficit have always resulted in an unacceptable level of false positives. New imaging technologies such as ultrasound and magnetic resonance now present more direct and more accurate methods for detecting increasing heart size, though they are considerably more expensive. While the detection of heart enlargement is, within itself, one predictor of an adverse outcome, the ECG “signals” of the proposed method have been found to be an independent predictor of a bad outcome, beyond increased heart size or mass. In addition, the method contained in the new invention includes multiple independent indicators of increased risk rather than one, all of which are incorporated in the risk score.

Accordingly, the present invention is directed to methods and devices for predicting risk of adverse cardiovascular events and/or mortality in an individual. In one aspect, the method for predicting risk of adverse cardiovascular events and/or mortality in an individual comprises the steps of: a) recording an electrocardiogram from the individual; b) analyzing the electrocardiogram to detect the presence of wave form elements; c) calculating a risk score based on the presence of the wave form elements; and d) predicting risk of adverse cardiovascular events and/or mortality based on the risk score. The step of recording an electrocardiogram may involve the use of any compatible recording instrument, including but not limited to an ECG recorder and analyzer, a computer, and a portable device such as a hand-held mobile device. The step of analyzing the electrocardiogram may be achieved by modifying the existing diagnostic computing module of a compatible recording instrument to detect the presence of specific wave form elements. The step of predicting risk of adverse cardiovascular events and/or mortality is based on the level of the risk score and may also comprise delivery of the score to a physician in an immediate report followed by the initiation or modification of a treatment program for the individual. Treatment programs may include, but are not limited to, medication, surgical procedures, regimes, or medical devices such as pacemakers.

In practice, the patient would receive an ECG upon the recommendation of the responsible physician, and the risk score would be generated by the computer within the instrument and delivered within the printed report normally delivered to the ordering physician, within a few minutes after completion of the recording. The physician would translate this score into an action plan for that individual patient. If “negative” (a low score), there would likely be no recommendation, but an elevated score would result in a recommendation appropriate to that patient. If the diagnosis is borderline hypertension, it is likely that an elevated score would trigger an appropriate antihypertensive drug. A striking elevation might trigger a more potent antihypertensive drug, plus others, such as statins, plus lifestyle changes. Follow-up risk scores would follow at yearly intervals, and further alterations in treatment would result from favorable (lower) or unfavorable (higher) values.

The generated risk score is an objective report, generated by the system, and not subject to bias or errors in reading intervals or magnitudes of waves in the ECG. It is recognized by most cardiologists that an automated determination of magnitude, width and direction of ECG events performed by the computational algorithms within the recording instrument are more consistent and accurate than those done by a human reader. In addition, these measurements avoid the fatigue, inattention and bias which often plague human efforts. Although a physician, with prior knowledge and practice, could score the ECG by manual calculations, this would require time and detailed effort to make the many required measurements, enter them into a formula, and calculate the score. It is not likely that this effort could be squeezed into the usual office visit, and the possibility of bias or error would be high.

In another aspect, calculation of the risk score is based on a longitudinal assessment of the presence of wave form elements and adverse cardiovascular events and mortality in a population-based cohort study. In a further aspect, the longitudinal assessment of wave form elements and adverse cardiovascular events and mortality in a population-based cohort study comprises analysis of the pattern of occurrence, intensity, and statistical response of wave form elements to predict specific adverse cardiovascular events and mortality. In a particular embodiment, the population-based cohort study is based on a population of over 10,000 individuals, more particularly about 15,000 individuals, followed over the course of over 10 years, particularly over 15 years, and more particularly about 20 years or more. In a further aspect, the longitudinal assessment of the specific score generated by each of the wave form elements, the total risk score, and the pattern of development and statistical response of these scores is used to predict specific types of cardiovascular disease.

In another aspect, total all-cause risk of mortality is predicted for an individual based on all identified wave form elements, and wherein each wave form element is rated and entered into the total risk score.

### Methods for Measuring Efficacy of Medications, Surgical Procedures, Dietary Regimes, or Treatment Programs

In another aspect, serial measurement of the risk score is used to measure the efficacy of a medication, a surgical procedure, a dietary regime, or a treatment program in the individual, wherein an increase in the risk score is indicative of poor efficacy and a reduction in the risk score is indicative of positive efficacy. In a further aspect, the total risk score is used to measure the efficacy of a medication, a surgical procedure, a dietary regime, or a treatment program in the individual, wherein an increase in the total risk score is indicative of poor efficacy and a reduction in the total risk score is indicative of positive efficacy. Treatment programs may include, but are not limited to, medication, surgical procedures, regimes, or medical devices such as pacemakers.

There is evidence that effective treatment of hypertension is able to reverse some of the ECG components used in the risk score. Sensitivity of the score in both directions (i.e. it moves up with time, patient age, and the progress of disease, but also down with effective therapy) can allow it to become another outcome endpoint in clinical trials of drugs and other cardiovascular interventions. This will provide a more responsive and sensitive indicator of effectiveness than the currently used tally of new cardiac events and/or cardiovascular mortality. This use of the invention will reduce the length and cost of controlled trials of drugs and other interventions.

There is also information that some classes of antihypertensive drugs may improve some of the ECG components of the risk score, while other classes of drugs, equally effective in lowering the blood pressure, do not. The risk score may predict some added and yet to be defined physiological effects of certain classes of drugs. Accordingly, the risk score will become an important added marker for this physiological effect, and an essential tool in further investigations of these drugs and their clinical usefulness (6).

It has been recently reported that patients who have undergone aortic valve replacement, either by the old direct surgical approach or by the newer transcatheter approach, have improvement in the ECG manifestations of left ventricular hypertrophy following a successful valve replacement. This risk score would provide a new objective measure of this improvement, and would provide a standardized method for quantitating this effect, allowing more objective comparisons of surgical outcomes from this procedure across multiple locations, surgeons, techniques, than would be possible from patient questionnaires and the opinions of surgical team members. The improvement in the ECG effects have been shown to correlate with other measures of improved function of the left ventricle after surgery (7).

It is likely that other interventions used today, or to be proposed in the future, will prove effective, and might have a similar effect, causing regression of the ECG effects which comprise the risk score, and therefore demonstrate an improved score. Possible examples include, but are not limited to alterations of diet or its components, physical activity, and weight loss. The effectiveness of these interventions in improving cardiovascular health could be objectively monitored by the use of the invention, and provide much needed objectivity.

### Methods for Predicting the Presence of Genetic Abnormalities

In another aspect, the pattern of occurrence, intensity, and statistical response of wave form elements are also used to predict the presence of one or more genetic abnormalities in the individual. In another aspect, the pattern of occurrence, intensity, and statistical response of wave form elements are also used to predict the presence or absence of one or more biomarkers in the individual. In this way, the methods of the present invention may serve as a proxy measure of these biomarkers. Among these biomarkers are genetic abnormalities and alterations of chemical constituents of the patient's blood or serum believed to be indicators of increased cardiovascular risk (i.e., the biomarkers are blood biomarkers).

As described in the Examples below, research results indicate that each of the six ECG components used in the ECG risk score are unique in their ability to predict specific cardiovascular outcomes, such as stroke, atrial fibrillation, heart failure and coronary artery disease. Detection and quantitation of these biologic markers now involve complex and expensive tests, which require days or weeks for results. Use of the automatic, immediate and relatively inexpensive ECG score as a proxy for these analyses is therefore an attractive option.

Without being bound by any particular theory or predicted mechanism, it is believed that each ECG feature is related to a specific genetic defect, located on one or more locations on specific chromosomes. Each of these genetic defects vary in impact on the person who has inherited them. Some produce trivial effects, and some are fatal. The observed ECG components within the risk score may have as their pathophysiologic basis one or more genetic defects, and thus enable the identification of these genetic defects and enable treatment of some before they produce heart disease or other bad effects. These genetic defects generate a chemical trail as they do their dirty work. For example, they might cause the cholesterol level to go up, or they might cause the blood pressure to elevate. In another example, the genetic defects may cause certain proteins (i.e. chemical biomarkers) to be generated in the heart or the kidneys, producing a high level of these elements which can be detected by analytic methods.

Accordingly, certain components of the electrocardiogram recorded from an individual may also be used to predict the presence of biomarker features in that individual, where the biomarker features are associated with adverse cardiovascular events, such as heart attacks, arrhythmias, congestive heart failure, strokes, and death. Genetic biomarker features include, but are not limited to, gene deletions, gene mutations, chromosome translocations, chromosome inversions, gene overexpression, gene underexpression, and post-translational modifications.

### Compatible Recording Instruments and Devices

In yet another aspect, one or more of any of the method steps disclosed herein are performed by a compatible recording instrument programmed to detect, quantitate, and analyze the wave form elements and calculate the risk score based upon the wave form elements. In a further aspect, the compatible recording instrument, including but not limited to an ECG recorder and analyzer, a computer, and a portable device such as a hand-held mobile device (9).

Compatible recording instruments can include any suitable type of electronic device, including a portable electronic device that the user may hold in hand (e.g., a portable media player or a cellular telephone), a larger portable electronic device (e.g., a laptop computer), or a substantially fixed electronic device. The electronic device may include software or hardware operative to process the output of one or more cardiac sensors to extract ECG components from the received output and calculate the ECG risk score as described herein.

To measure the ECG components, the compatible recording device can include one or more sensors embedded in the device. The one or more sensors can include leads for receiving electrical signals from the user's heart. For example, the one or more sensors can include leads associated with the user's left and right sides, and a lead associated with the “ground.” To provide an electrical signal from the user to the processing circuitry, the leads can be exposed such that the user may directly contact the leads, or may instead or in addition be coupled to an electrically conductive portion of the device enclosure (e.g., a metallic bezel or housing forming the exterior of the device), or utilize a “harness” of wires and electrodes designed to properly connect the locations on the body surface to the electronic circuitry.

In yet another aspect, the compatible recording instrument is able to interact with other recording or analytic devices.

In a further aspect, a computer readable medium is provided programmed to perform one or more of any of the method steps disclosed herein. Any suitable computer useable medium may be utilized for software aspects of the invention. The computer-usable or computer-readable medium may be, for example but not limited to, an electronic, magnetic, optical, electromagnetic, infrared, or semiconductor system, apparatus, device, or propagation medium. The computer readable medium may include transitory and/or non-transitory embodiments. More specific examples (a non-exhaustive list) of the computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

### GENERAL DEFINITIONS

The presently disclosed subject matter is described with specificity to meet statutory requirements. However, the description itself is not intended to limit the scope of this patent. Rather, the inventor has contemplated that the claimed subject matter might also be embodied in other ways, to include different steps or elements similar to the ones described in this document, in conjunction with other present or future technologies (e.g., the calculation of the validated risk score via a vectorcardiogram, the inclusion of additional electrocardiogram components, an alternative method of statistical weighing, and the like).

Although the six ECG components used for the risk score calculations have been known for years, there has been no concerted effort to identify other components which might join these six and improve the predictive ability of the risk score. There are other ECG components which have been noted to correlate with a higher mortality or a higher incidence of CVD, but have not been tested to sufficiently to document that their inclusion would augment the risk predictive ability of the original six. To be included they would need to add to the ability of the original six, indicating that they are testing for new pathophysiological states, not recognized by the original group. If found to meet this requirement they may be included as an added ECG element. It is also possible that the new candidate could replace a current component, by demonstrating that it duplicates the predictive pattern of an earlier component, but at a higher level of sensitivity. It is also possible that a new component might duplicate the risk predictive pattern of an earlier component, but prove easier or cheaper to calculate than the earlier component.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Moreover, although the term “step” may be used herein to connote different aspects of methods employed, the term should not be interpreted as implying any particular order among or between various steps herein disclosed unless and except when the order of individual steps is explicitly described.

Although the foregoing subject matter has been described in some detail by way of illustration and example for purposes of clarity of understanding, it will be understood by those skilled in the art that certain changes and modifications can be practiced within the scope of the appended claims.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to practice the methods of the present invention.

### Example 1

This study aimed at the quantitation and better understanding of the prognostic significance of the ECG features of the R-E Score as a predictor of all-cause mortality.

### Methods

The population used for this analysis included 15,792 participants, aged 45 to 64 years who participated in the Atherosclerosis Risk in Communities (ARIC) Study. This cohort was recruited and first examined in 1987-1989 from 4 US communities. The ARIC study and its methods have been described elsewhere (8). Follow-up visits were carried out in 1990-1992 (93% return rate), 1993-1995 (86%), 1996-1998 (80%) and 2011-2013 (65%).

For the purpose of this analysis, we excluded 808 participants: 196 had no ECG, 136 had ECGs of inadequate quality, 429 had an external pacemaker, Wolff-Parkinson-White pattern or complete bundle branch blocks, and 47 were neither African-American nor white in ethnic origin.

Electrocardiography:

At each study exam, a standard supine 12-lead resting ECG was recorded with a MAC PC Personal Cardiograph (Marquette Electronics, Milwaukee, Wis., USA) and transmitted to the ARIC ECG Reading Center (EPICARE Center, Wake Forest School of Medicine, Winston Salem, N.C.) for automatic coding. ECGs were automatically processed using Marquette 12-SL Version 2001 (GE, Milwaukee, Wis., USA). R-E score was calculated from 6 ECG features with a specific value of points for each feature as follows: R or S wave in any limb lead ≧2 my, or S wave in V1 or V2≧3 my., or R wave in V5 or V6≧3 my (3 points); P terminal force defined as terminal negativity of P wave in V1≧0.10 mV in depth and ≧0.04 msec in duration (3 points); left ventricular strain defined as ST segment and T wave in opposite direction to QRS in V5 or V6, without digitalis (3 points); left axis deviation defined as QRS axis ≦−30 degrees (2 points); QRS duration ≧0.09 msec (1 point); and intrinsicoid deflection in V5 or V6≧0.05 msec (1 point).

Covariates:

Baseline age, sex, race, education level, income and smoking status were determined by self-report. Body mass index (BMI) at baseline was calculated as weight (in kilograms) divided by height (in meters) squared. Blood samples were obtained after an 8-hour fasting period. Diabetes was defined as a fasting glucose level ≧126 mg/dL (or non-fasting glucose ≧200 mg/dL), a self-reported physician diagnosis of diabetes, or use of diabetes medications. Hypertension was defined as systolic blood pressure ≧140 mmHg, diastolic blood pressure ≧90 mmHg, or use of blood pressure lowering medications. Prevalent CVD was identified by self-reported history or a previous physician diagnosis.

Statistical Analysis:

Baseline R-E scores were calculated for all participants and various baseline characteristics of the population were tabulated and compared across increasing levels of the R-E score, grouped as follows: score=0, 1-3, 4, and >=5. Incidence rates of all-cause mortality per 1000 person-years in each of the R-E score levels occurred during follow up (from visit 2 until December 2010) were calculated, and Kaplan-Meier survival curves were plotted to compare event-free survival across these ascending score levels.

Cox proportional hazards analysis was used to examine the association between R-E score and all-cause mortality in a series of models as follows: Model 1, unadjusted; Model 2, adjusted for age, sex, and race; and Model 3. adjusted for the model 2 variables plus: field center, BMI, systolic blood pressure, smoking status, education, hypertension, diabetes mellitus, cardiovascular disease status, family history of CHD, ratio of total cholesterol/high-density lipoprotein, blood glucose, and serum creatinine at baseline. In these models, R-E score 0 was the reference group and risk of mortality was evaluated in 3 groups of R-E score (1-3, 4, and >=5).

Using similar models, the association between change in the score between the baseline visit and the first return visit with mortality was also examined. The group that exhibited no change served as the reference group for this analysis.

The risk of mortality was also calculated for each of the six components of the score: P-terminal force in V1, QRS voltage, left axis deviation, QRS duration, intrinsicoid deflection time, and ST/T abnormalities (left ventricular strain). Each of these components was evaluated separately as present/absent at the baseline visit, with the absent value group as the reference group. Models were adjusted in a similar fashion as mentioned above but with an additional model 4 in which the 6 components were added to those in model 3.

Statistical significance for all analyses was p<0.05. Analyses were conducted using SAS 9.2 (SAS Institute, Cary, N.C.).

### Results

A total of 14,984 participants (age 54.1±5.8 years; 55.8% females; 26.9% African Americans) were included in this analysis. The baseline prevalence of R-E score was as follows: R-E=0 in 6342 participants, 1-3 in 8017 participants, 4 in 416 participants and 5 or more in 209 participants. Table 1 shows the participants characteristics across levels of R-E score. Participant characteristics found to be associated with increasing levels of R-E score were age, body mass index, systolic blood pressure, African-American ethnicity, male sex, education level, smoking, diabetes, total cholesterol, hypertension, use of blood-pressure lowering drugs, and history of coronary heart disease. On the other hand, family history of coronary heart disease and statin use did not differ across R-E levels.

TABLE 1
Baseline participant characteristics stratified by levels of Romhilt-Estes score

| | Score = 0 | Score ≦3 | Score = 4 | Score ≧5 | |
|---|---|---|---|---|---|
| Mean (SD) or % | n = 6342 | n = 8017 | n = 416 | n = 209 | P value* |
| Age (years) | 54 (5.7) | 54 (5.7) | 56 (5.7) | 56 (5.5) | <.0001 |
| Body mass index (kg/m²) | 27 (5.5) | 28 (5.3) | 28 (5.4) | 28 (5.0) | <.0001 |
| Systolic blood pressure (mmHg) | 120 (18.4) | 121 (18.0) | 134 (24.3) | 137 (29.0) | <.0001 |
| Total cholesterol (mg/dL) | 216 (42.3) | 214 (41.6) | 218 (42.6) | 213 (49.5) | 0.003 |
| Women (%) | 74.4 | 42.5 | 38.7 | 35.4 | <.0001 |
| African-American (%) | 29.1 | 23.4 | 49.0 | 49.8 | <.0001 |
| Education (≦high school) (%) | 56.9 | 54.8 | 63.2 | 66.0 | <.0001 |
| Smoke (current) (%) | 26.9 | 24.8 | 34.0 | 37.3 | <.0001 |
| Diabetes (%) | 10.9 | 11.7 | 20.9 | 24.5 | <.0001 |
| Hypertension (%) | 30.7 | 35.7 | 59.4 | 70.7 | <.0001 |
| Use of blood pressure lowering drugs (%) | 26.5 | 31.2 | 53.9 | 64.1 | <.0001 |
| Statin use (%) | 0.5 | 0.6 | 1.2 | 1.0 | 0.280 |
| History of coronary heart disease (%) | 2.2 | 4.8 | 18.5 | 35.6 | <.0001 |
| Family history of coronary heart disease (%) | 39.4 | 39.7 | 36.8 | 40.7 | 0.669 |

*Statistical significance for categorical variables tested using the chi-square method and for continuous variables the Kruskal-Wallis was used.

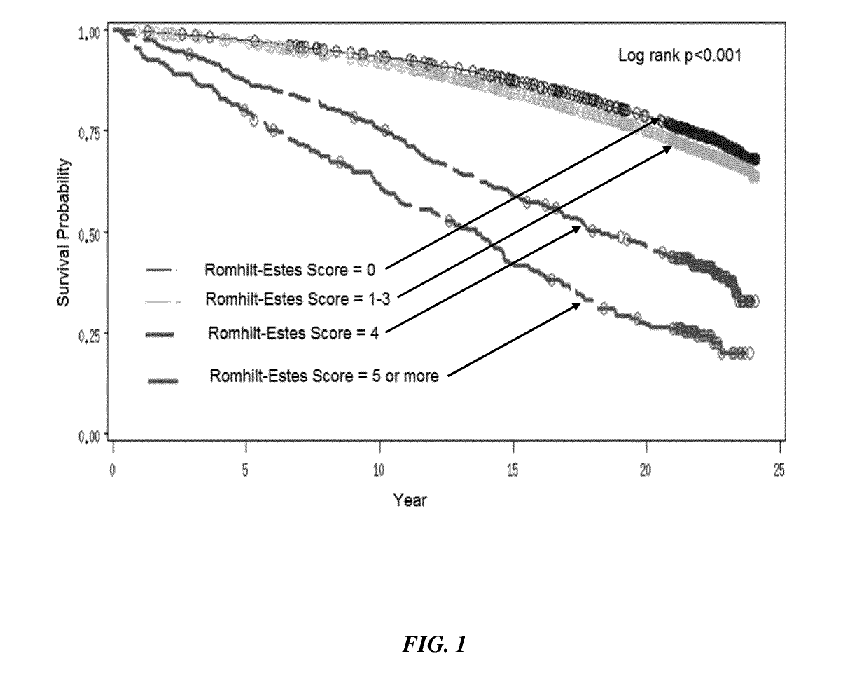

During a median follow up of 21.7 years, 4549 all-cause mortality events occurred. The incidence rate of all-cause mortality was lowest in those with R-E score=0 and highest in those with R-E score ≧5 (Incidence rates per 1000 person years=13.8, 16.2, 38.8, and 60.5 in participants with R-E score=0, 1-3, 4, and ≧5, respectively). FIG. 1 shows the Kaplan Meier survival curves by levels of R-E score.

The risk of all-cause mortality was increasing as the levels of the R-E score increased reaching over four times in those with R-E score ≧5 compared to those with R-E score=0. This pattern of associations remained significant even after adjustment for participant characteristics (Table 2).

TABLE 2
Prediction of risk for all-cause mortality by Romhilt/Estes score present at baseline

| | | Event rate/ | | | | | | |
|---|---|---|---|---|---|---|---|---|
| | | 1000 person | | | | | | |
| | N | years | Model-1 | p-value | Model-2 | p-value | Model-3 | p-value |
| Score = 0 | 6342 | 13.8 | 1 (ref) | | 1 (ref) | | 1 (ref) | |
| Score 1-3 | 8017 | 16.2 | 1.18 (1.11-1.26) | <.0001 | 1.05 (0.99-1.12) | 0.126 | 1.00 (0.93-1.07) | 0.937 |
| Score = 4 | 416 | 38.8 | 2.67 (2.34-3.05) | <.0001 | 2.06 (1.80-2.36) | <.0001 | 1.60 (1.39-1.84) | <.0001 |
| Score ≧5 | 209 | 60.5 | 4.50 (3.82-5.31) | <.0001 | 3.50 (2.96-4.14) | <.0001 | 2.08 (1.75-2.48) | <.0001 |

Model-1: Unadjusted
Model-2: Adjusted for age, sex and race;
Model-3: Adjusted for demographic and clinical variables of age, sex, race, field center, body mass index, systolic blood pressure, smoking status, education, hypertension, diabetes mellitus, cardiovascular disease status, family history of CHD, ratio of total cholesterol/high-density lipoprotein, blood glucose, and serum creatinine at baseline

Table 3 shows the risk of mortality associated with each of the six individual components of the R-E score. As shown, four of the six ECG components of the score (P-terminal force in V1, QRS amplitude, LV strain, and intrinsicoid deflection) were predictive of all-cause mortality in the fully adjusted models which also included all the six components together while two of the components were not (left axis deviation and prolonged QRS duration). Differences in the strengths of the associations between the individual components of the score and mortality were also observed.

TABLE 3
Baseline Romhilt/Estes score components and risk for all-cause mortality

| | Event rate/1000 | | | | |
|---|---|---|---|---|---|
| | person years | | | | |
| | Absent | Present | Model | HR (95% CI) | P-value |
| R or S wave in any limb lead ≧2 mv, or | 15.8 | 37.1 | Model 1^{a} | 2.48 (2.09-2.94) | <.0001 |
| S wave in V1 or V2 ≧3 mv, or R wave in | | | Model 2^{b} | 1.81 (1.52-2.15) | <.0001 |
| V5 or V6 ≧3 mv. (n = 236) (present vs. | | | Model 3^{c} | 1.43 (1.19-1.71) | 0.0001 |
| absent) | | | Model 4^{d} | 1.21 (1.01-1.46) | 0.0436 |
| Left atrial enlargement: terminal | 15.8 | 45.2 | Model 1^{a} | 2.60 (2.17-3.10) | <.0001 |
| negativity of P wave in V1 ≧0.10 mV in | | | Model 2^{b} | 2.35 (1.97-2.81) | <.0001 |
| depth and ≧0.04 msec in duration | | | Model 3^{c} | 1.74 (1.45-2.09) | <.0001 |
| (n = 193) (present vs. absent) | | | Model 4^{d} | 1.62 (1.34-1.95) | <.0001 |
| Left ventricular strain: ST segment and | 15.2 | 46.4 | Model 1^{a} | 2.90 (2.60-3.23) | <.0001 |
| T wave in opposite direction | | | Model 2^{b} | 2.64 (2.36-2.95) | <.0001 |
| to QRS in V5 or V6, without digitalis | | | Model 3^{c} | 1.83 (1.63-2.06) | <.0001 |
| (n = 529) (present vs. absent) | | | Model 4^{d} | 1.72 (1.53-1.94) | <.0001 |
| Left axis deviation: ≦(−30) degrees | 15.7 | 26.5 | Model 1^{a} | 1.49 (1.32-1.69) | <.0001 |
| (n = 593) (present vs. absent) | | | Model 2^{b} | 1.34 (1.18-1.52) | <.0001 |
| | | | Model 3^{c} | 1.14 (1.01-1.30) | 0.0373 |
| | | | Model 4^{d} | 1.09 (0.96-1.23) | 0.2075 |
| QRS duration ≧0.09 msec. (n = 8194) | 14.7 | 17.2 | Model 1^{a} | 1.19 (1.12-1.26) | <.0001 |
| (present vs. absent) | | | Model 2^{b} | 1.04 (0.98-1.11) | 0.2161 |
| | | | Model 3^{c} | 1.00 (0.94-1.06) | 0.9670 |
| | | | Model 4^{d} | 0.97 (0.91-1.03) | 0.2846 |
| Intrinsicoid deflection in V5 or V6 ≧0.05 msec. | 15.8 | 25.1 | Model 1^{a} | 1.75 (1.52-2.00) | <.0001 |
| (n = 504) (present vs. absent) | | | Model 2^{b} | 1.67 (1.45-1.92) | <.0001 |
| | | | Model 3^{c} | 1.44 (1.25-1.66) | <.0001 |
| | | | Model 4^{d} | 1.38 (1.20-1.60) | <.0001 |

^{a}Model-1: Unadjusted
^{b}Model-2: Adjusted for age, sex and race;
^{c}Model-3: Adjusted for demographic and clinical variables of age, sex, race, field center, body mass index, systolic blood pressure, smoking status, education, hypertension, diabetes mellitus, cardiovascular disease status, family history of CHD, ratio of total cholesterol/high-density lipoprotein, blood glucose, and serum creatinine at baseline
^{d}Model-4: Adjusted for all demographic and clinical variables in Model 3 plus (instead of and) the total of all six components.

In an attempt to duplicate the clinical situation of a patient being followed by his/her clinician and who develops a higher score between visits, Table 4 is presented. This table presents the risk for all-cause mortality associated with a change in R-E score between the baseline and first follow up visit, using the “no change” group as the reference. As seen, there is a steady rise in event rate with each point increase in the score.

TABLE 4
Change in Romhilt/Estes score over time and risk for all-cause mortality

| | | Event rate | | | | | | |
|---|---|---|---|---|---|---|---|---|
| | N | (%) | Model-1 | p-value | Model-2 | p-value | Model-3 | p-value |
| change = 0 | 4625 | 22.8 | 1.00 (ref) | | 1.00 (ref) | | 1.00 (ref) | |
| change = 1 | 5775 | 27.6 | 1.22 (1.11-1.30) | <.0001 | 1.19 (1.10-1.28) | <.0001 | 1.19 (1.10-1.28) | <.0001 |
| change = 2 | 2298 | 30.8 | 1.35 (1.22-1.48) | <.0001 | 1.31 (1.19-1.44) | <.0001 | 1.26 (1.14-1.39) | <.0001 |
| change = 3 | 714 | 46.1 | 2.23 (1.97-2.52) | <.0001 | 2.13 (1.88-2.41) | <.0001 | 1.74 (1.53-1.98) | <.0001 |
| change ≧4 | 188 | 53.7 | 2.58 (2.11-3.17) | <.0001 | 2.53 (2.06-3.11) | <.0001 | 2.12 (1.71-2.61) | <.0001 |

Model-1: Unadjusted
Model-2: Adjusted for age, sex and race;
Model-3: Adjusted for demographic and clinical variables of age, sex, race, field center, body mass index, systolic blood pressure, smoking status, education, hypertension, diabetes mellitus, cardiovascular disease status, family history of CHD, ratio of total cholesterol/high-density lipoprotein, blood glucose, and serum creatinine at baseline

### Discussion

When a clinician orders an ECG in the course of treating a patient, he/she engages an unexpressed promise to the patient that the information obtained might lead to information favorable to treatment. This clinician is also obliged to use evidence-based treatment decisions to assure that the patient's time and money are well spent. It is the lack of such evidence that has led the US Preventive Services Task Force not to recommend the use of the ECG as a routine screening tool for coronary heart disease in asymptomatic adults (10). The use of the ECG as a follow up tool in the course of treatment of hypertension and other forms of cardiovascular disease similarly lacks solid evidence of its usefulness as a screening tool. It is not that electrocardiographers have been idle over the past decades. Instead, most of their efforts have been in trying to improve the precision of the ECG in predicting increased LV mass/LVH.

Cardiologists of the mid-20th century recognized that clinical signs of LVH were an adverse development, and the ECG was seen as a noninvasive tool for earlier detection of this development, at a time when there was no noninvasive alternative available, other than a chest roentgenogram. Therefore, the objective of research was in developing more sensitive and precise techniques of obtaining an ECG “diagnosis” of LVH. Information about the LV mass is now easily provided by imaging techniques, such as echo and MRI, and these techniques are clearly better at the task than the ECG. Only recently has the focus of research begun to focus on other capabilities of the ECG. Some groups, such as the Working Group on the Electrocardiographic Diagnosis of Left Ventricular Hypertrophy (10) have urged that the ECG be primarily used for prediction of increased risk, and that the search for better ECG-LVH methods be abandoned. It was also suggested that the term ECG-LVH should be replaced by a more appropriate term, to fit this revised purpose.

Two facts have emerged over the past several decades which provide added impetus for refocusing electrocardiographic research. One is the demonstration that the same ECG changes we once used to “diagnose” an increase in LV mass have an ability to predict an adverse course of the underlying disease, independent of LV mass (11). The other is the demonstration that these changes are potentially reversible, meaning that their disappearance signals a favorable turn in the course of the underlying illness (12). If these early observations can be validated, quantitated and expanded, we would likely have evidence adequate to support the use of the ECG as a reliable guide to treatment. This guide could indicate the need for change in treatment, and serve as an added incentive to the patient as he/she adapts to tightened therapy or altered lifestyle.

As discussed above, the R-E score, as originally proposed for the “diagnosis” of LVH, also predicts an increase in all-cause mortality at a highly significant level, and a further increase in the point score from one visit to the next is even more striking as an indicator of increased risk. The conclusion is that the R-E score, as such, is a powerful predictive tool for all-cause mortality.

In addition, this analysis shows that the majority of the individual ECG components that comprise the R-E score are independently predictive of all-cause mortality. Specifically, the P-terminal force, ST-T changes of left ventricular strain, and the duration of the “intrinsicoid deflection” are all strong predictors of all-cause mortality. Interestingly, QRS amplitude, the component given highest value in most ECG-LVH criteria, is the least powerful component of the set.

Each of the components of the R-E score represents a different variation in electrical events within the myocardium, but we have little information about the precise alterations that underlie these ECG “patterns”. It is possible that each of the four predicative components signals a different electrical event within the myocardium, and a different ability to predict cardiovascular outcomes. It is also likely that other ECG patterns will prove to have the same ability to predict adverse cardiovascular events, and will join the above set of four. These and other questions are subjects for future investigations.

The results of this study clearly imply potential usefulness of the ECG as a predictive tool in clinical care of patients with cardiovascular disease. The set identified in this study are those generated by autopsy and hemodynamic studies almost a half century ago, for another purpose. It seems likely that they can be refined and clarified by further study, and made even more powerful. These findings make the objective of a validated, non-invasive clinical tool a likely possibility, and worthy of further study.

Conclusions:

The R-E score is highly predictive of all-cause mortality, both as a single baseline score, and as an increasing score over time. The six individual ECG components of the R-E score contain four components with independent predictive ability.

### Example 2

As shown in Example 1, the electrocardiographic Romhilt-Estes Point Score (R-E Score) is associated with an increased risk of all-cause mortality in the general population, and that different score components show different predictive abilities (5). We sought to extend our previous work that examined the association between R-E score and all-cause mortality to cardiovascular disease (CVD) outcomes. We hypothesized that different components of the R-E score would be associated with different CVD outcomes (heart failure (HF), coronary heart disease (CHD), stroke, and a composite of these outcomes referred herein as composite CVD). Without being bound by any particular theory, it is believed that ventricular hypertrophy and the ECG changes historically used to indicate its presence are independent, but related phenomena. That is to say, the components of the R-E Score are distinct electrical characteristics involving both atrial and ventricular, and both depolarization and repolarization phases of myocardial electrical activity, and that they are associated with different clinical outcomes. This hypothesis was examined using data from the Atherosclerosis Risk in Communities (ARIC) Study, one of the largest biracial longitudinal cohort studies in the United States (US).

### Methods

The Atherosclerosis Risk in Communities (ARIC) Study includes 15,792 participants, aged 45 to 64 years, from four US communities: Forsyth County, N.C., Jackson, Miss., Minneapolis, Minn., and Washington County, Md. The subjects were selected by probability sampling in three communities. In Jackson, Miss. only blacks are included in the sample. The selection methods and study details have been described elsewhere (5). The first examinations were begun in 1986, and the first cycle of the study completed in 1989. Follow-up visits were carried out in 1990-1992 (93% return rate), 1993-1995 (86%), 1996-1998 (80%) and 2011-2013 (65%).

ARIC studies are approved by the institutional review boards of the participating community study sites. All participants also provided written informed consent.

For this analysis, we excluded 196 who had no ECG, 136 with ECGs of inadequate quality, 429 with an external pacemaker, Wolff-Parkinson-White pattern or complete bundle branch block, and 47 who were not African-American or white in ethnic origin. Also, 1,723 participants with baseline CVD, defined as coronary heart disease (CHD), heart failure (HF), stroke or atrial fibrillation (AF), were also eliminated. After all exclusions, 13,261 participants remained and are included in this analysis.

Electrocardiography:

At each study exam, a standard supine 12-lead resting ECG was recorded with a MAC PC Personal Cardiograph (Marquette Electronics, Milwaukee, Wis., USA) and transmitted to the ARIC ECG Reading Center (Epidemiological Cardiology Research Center (EPICARE), Wake Forest School of Medicine, Winston Salem, N.C.) for reading and coding.

ECGs were automatically processed using Marquette 12-SL Version 2001 (GE, Milwaukee, Wis., USA). R-E score was calculated from six ECG features with a specific value of points for each feature as follows: QRSAMP-R or S wave in any limb lead ≧2 mV, or S wave in V1 or V2≧3 mV., or R wave in V5 or V6≧3 mV. (3 points); PTFV1-P terminal force defined as terminal negativity of P wave in V1≧0.10 mV in depth and ≧0.04 sec in duration (3 points); LVSTR-left ventricular strain defined as ST segment and T wave in opposite direction to QRS in V5 or V6, without digitalis (3 points); LAXDEV-left axis deviation defined as QRS axis ≦−30 degrees (2 points); QRSDUR-QRS duration ≧0.09 sec (1 point); and INTRNS-intrinsicoid deflection duration in V5 or V6≧0.05 sec (1 point).

Cardiovascular Outcomes:

The outcomes of stroke, heart failure, and CHD were determined by physicians, using validated adjudication protocols. Stroke was defined as sudden neurologic insult of = or ≧24 hour duration or a neurologic insult associated with death without evidence of a non-stroke cause of death (14). Stroke events were ascertained from surveillance of ARIC participant hospitalizations using ICD-9 codes 430-438 through 1997 and codes 430-436 thereafter. Strokes were classified by physician review and computer algorithm with standardized criteria and determined as hemorrhagic or ischemic.

Heart failure was ascertained by review of hospitalization records and death certificates for a heart failure diagnosis. Specifically, incident cases with an ICD-9 code of 428 (428.0-428.9) or ICD Tenth Revision 150 were classified as heart failure (15). CHD was determined using study surveillance and adjudicated as described (16,17). Symptoms, biomarkers, and electrocardiography were incorporated into a computerized algorithm. Disagreement between discharge coding and computer algorithm were adjudicated by the ARIC Mortality and Morbidity Classification Committee. For the present analysis, CHD was defined as definite or probable myocardial infarction or definite fatal CHD. Incident CVD was defined as the first occurrence of any of a composite of CHD, stroke or HF.

Covariates:

Baseline age, sex, race, education level, income and smoking status were determined by self-report. Body mass index (BMI) at baseline was calculated as weight (in kilograms) divided by height (in meters) squared. Blood samples were obtained after an 8-hour fasting period. Diabetes was defined as a fasting glucose level ≧126 mg/dL (or non-fasting glucose ≧200 mg/dL), a self-reported physician diagnosis of diabetes, or use of diabetes medications. Hypertension was defined as systolic blood pressure ≧140 mmHg, diastolic blood pressure ≧90 mmHg or use of blood pressure lowering medications. Prevalent CVD was identified by self-reported history or a previous physician diagnosis.

Statistical Analysis:

Baseline R-E scores for all participants were calculated, and various baseline characteristics of the population were tabulated and compared across increasing score levels, grouped as follows: score=0, 1-3, and ≧4. Incidence rates of new CVD per 1000 person-years in each of the three R-E score levels occurring during follow-up (from Visit 1 to December 2010) were calculated.

Cox proportional hazards analysis was used to examine the association between R-E score and each of the outcomes (CVD, CHD, HF, and stroke) in a series of models as follows: Model 1, adjusted for age, sex, and race; and Model 2. adjusted for the Model 1 variables plus: field center, BMI, systolic blood pressure, smoking status, education, hypertension, diabetes mellitus, family history of CHD, total cholesterol/high-density lipoprotein ratio, blood glucose, serum creatinine and serum uric acid. In these models, R-E score 0 was the reference group and risk of new CVD was evaluated across the three groupings of the R-E score (0, 1-3, ≧4).

The associations between each of the six components of the R-E score: QRSAMP, PTFV1, LVSTR, LAXDEV, QRSDUR, and INTRNS, as a baseline ECG finding, with different CVD outcomes were also examined. Each of the R-E score components was evaluated separately as present/absent at the baseline visit, with the absent value group as the reference group. Models were adjusted in a similar fashion as mentioned above, but with an additional model 3 in which adjustments for each and all of the six components were added to those present in model 2.

We examined the assumption of proportional hazards by computation of Schoenfeld residuals and inspection of log(−log(survival function)) curves, and they were met. Statistical significance for all analyses was p<0.05. Analyses were conducted using SAS 9.3 (SAS Institute, Cary, N.C.)

### Results

A total of 13,261 participants (age 53.8±5.3 years; 56.9% females; 26.3% African Americans) were included in this analysis. Table 5 shows the participant characteristics across different levels of the R-E score. Participants characteristics found to be positively associated with increasing levels of R-E score were age, African-American ethnicity, male sex, body mass index, systolic blood pressure, total cholesterol, blood glucose, serum creatinine, uric acid, lower education level, smoking, diabetes, hypertension, and use of blood-pressure lowering drugs.

TABLE 5
Baseline participants characteristics stratified by levels of Romhilt/Estes score

| | Score = 0 | Score ≦3 | Score ≧4 | |
|---|---|---|---|---|
| N = 13,261 | n = 5860 | n = 7037 | n = 364 | P value |
| Age (years) | 54 (5.8) | 54 (5.7) | 56 (5.8) | <.0001 |
| Body mass index (kg/m2) | 27 (5.4) | 28 (5.1) | 27 (5.3) | <.0001 |
| Systolic blood pressure (mmHg) | 119 (18.1) | 121 (17.8) | 137 (25.6) | <.0001 |
| Total cholesterol (mg/dL) | 216 (42.1) | 213 (41.0) | 213 (40.3) | 0.0012 |
| High-density lipoprotein (mg/dL) | 55 (17.2) | 50 (16.7) | 53 (18.4) | <.0001 |
| Blood glucose (mg/dL) | 107 (38.9) | 108 (37.2) | 113 (50.9) | 0.0043 |
| Serum creatinine (mg/dL) | 1.1 (0.3) | 1.1 (0.2) | 1.3 (1.2) | <.0001 |
| Uric acid (mg/dL) | 5.6 (1.4) | 6.2 (1.6) | 6.5 (1.7) | <.0001 |
| Women (%) | 74.9 | 42.7 | 42.0 | <.0001 |
| African-American (%) | 28.5 | 23.0 | 54.1 | <.0001 |
| Education (≦high school) (%) | 56.1 | 53.5 | 62.6 | 0.0001 |
| Smoke (current) (%) | 26.5 | 24.5 | 33.9 | <.0001 |
| Diabetes (%) | 9.9 | 10.2 | 16.1 | 0.0009 |
| Hypertension (%) | 28.2 | 32.2 | 62.2 | <.0001 |
| Use of blood pressure lowering drugs (%) | 22.7 | 25.8 | 45.9 | <.0001 |
| Family history of coronary heart disease (%) | 39.1 | 39.1 | 34.3 | 0.1859 |

During a median follow-up of 21.8 years, 3,579, 2,205, 1,814, and 731 CVD, CHD, HF, and stroke events, respectively, occurred. The incidence rates of these outcomes were lowest in those with R-E score=0 and highest in those with an R-E score ≧4 points. R-E score ≧4 points (compared to R-E score=0 point) was significantly associated with increased risk of CVD, CHD, HF and stroke after adjustment for common CVD risk factors and potential confounders (Table 6).

TABLE 6
Baseline Romhilt-Estes score and risk of incident cardiovascular disease

| Event rate | | Model-1 | | Model-2 | |
|---|---|---|---|---|---|
| n/N | % | HR (95% CI) | P-value | HR (95% CI) | P-value |

Incident Cardiovascular Disease

| | | | | | | |
|---|---|---|---|---|---|---|
| Score = 0 | 1375/5860 | 23.5 | 1.00 (ref) | | 1.00 (ref) | |
| Score 1-3 | 2030/7037 | 28.9 | 1.10 (1.02-1.18) | 0.0098 | 1.07 (0.99-1.15) | 0.0879 |
| Score ≧4 | 174/364 | 47.8 | 2.03 (1.73-2.39) | <.0001 | 1.66 (1.41-1.96) | <.0001 |

Incident Coronary Heart Disease

| | | | | | | |
|---|---|---|---|---|---|---|
| Score = 0 | 788/5860 | 13.5 | 1.00 (ref) | | 1.00 (ref) | |
| Score 1-3 | 1316/7037 | 18.7 | 1.10 (1.00-1.21) | 0.0510 | 1.09 (0.99-1.19) | 0.0874 |
| Score ≧4 | 101/364 | 27.8 | 1.94 (1.57-2.39) | <.0001 | 1.66 (1.34-2.07) | <.0001 |

Incident Heart Failure

| | | | | | | |
|---|---|---|---|---|---|---|
| Score = 0 | 731/5793 | 12.6 | 1.00 (ref) | | 1.00 (ref) | |
| Score 1-3 | 970/6943 | 14.0 | 1.12 (1.01-1.24) | 0.0289 | 1.02 (0.92-1.13) | 0.7678 |
| Score ≧4 | 113/356 | 31.7 | 2.52 (2.06-3.08) | <.0001 | 1.97 (1.60-2.43) | <.0001 |

Incident Stroke

| | | | | | | |
|---|---|---|---|---|---|---|
| Score = 0 | 304/5860 | 5.2 | 1.00 (ref) | | 1.00 (ref) | |
| Score 1-3 | 381/7037 | 5.4 | 1.08 (0.86-1.18) | 0.9147 | 0.96 (0.81-1.12) | 0.5779 |
| Score ≧4 | 46/364 | 12.6 | 2.06 (1.50-2.82) | <.0001 | 1.49 (1.07-2.07) | 0.0178 |

Model-1: Adjusted for age, sex and race;
Model-2: Adjusted for variables in model 1 plus study site, body mass index, systolic blood pressure, smoking status, education, hypertension, diabetes mellitus, cardiovascular disease status, family history of coronary heart disease, ratio of total cholesterol/high-density lipoprotein, blood glucose, serum creatinine, and uric acid (all at baseline).

Table 7 shows the associations between the individual components of the R-E score and incident CVD outcomes. As shown, all of the six R-E score were predictive of CVD events in the demographic adjusted model. However, after further adjustment for CVD risk factors and potential confounders (model 2) or when the six components were entered together in the model (model 3), only PTFV1, LVSTR and LAXDEV retained their significant associations with CVD.

TABLE 7
Baseline Romhilt-Estes score components and risk of incident cardiovascular disease

| | Event rate (%) | | Model-1^{a} | Model-2^{b} | Model-3^{c} |
|---|---|---|---|---|---|
| | Absent | Present | HR (95% CI) | HR (95% CI) | HR (95% CI) |
| QRSAMP | 26.8 | 40.8 | 1.40 (1.10-1.77)^{‡} | 1.17 (0.92-1.50) | 1.04 (0.81-1.33) |
| PTFV1 | 26.9 | 38.3 | 1.51 (1.12-2.03)^{‡} | 1.41 (1.03-1.91)^{†} | 1.42 (1.04-1.93)^{†} |
| LVSTR | 26.5 | 48.0 | 2.28 (1.93-2.70)^{§} | 1.65 (1.38-1.96)^{§} | 1.62 (1.36-1.94)^{§} |
| LAXDEV | 26.6 | 39.5 | 1.34 (1.16-1.56)^{§} | 1.21 (1.04-1.42)^{†} | 1.19 (1.02-1.39)^{†} |
| QRSDUR | 24.2 | 29.4 | 1.09 (1.01-1.16)^{†} | 1.07 (1.00-1.15) | 1.06 (0.98-1.14) |
| INTRNS | 26.8 | 32.7 | 1.26 (1.04-1.52)^{†} | 1.20 (0.99-1.45) | 1.17 (0.96-1.41) |

^{†}Denotes P < 0.05;
^{‡}P < 0.01;
^{§}P < 0.001 for P values of hazard ratios
^{a}Model-1: Adjusted for age, sex and race;
^{b}Model-2: Adjusted for variables in model 1 plus field center, body mass index, systolic blood pressure, smoking status, education, hypertension, diabetes mellitus, family history of coronary heart disease, ratio of total cholesterol/high-density lipoprotein, blood glucose, serum creatinine, and uric acid (all at baseline)
^{c}Model-3: Adjusted for variables in Model 2 plus all of the six R-E score components.
QRSAMP—R or S wave in any limb lead ≧2.0 mV, or S wave in V1 or V2 ≧3.0 mV, or R wave in V5 or V6 ≧3.0 mV;
PTFV1—P terminal force defined as terminal negativity of P wave in V1 ≧0.10 mV in depth and ≧0.04 sec in duration;
LVSTR—Left ventricular strain defined as ST segment and T wave in opposite direction to QRS in V5 or V6, without digitalis;
LAXDEV—Left axis deviation defined as QRS axis ≦−30 degrees;
QRSDUR—QRS duration ≧0.09 sec;
INTRNS—Intrinsicoid deflection duration in V5 or V6 ≧0.05 sec;

Table 8 shows the associations between each component of the R-E score at baseline with individual CVD outcomes (HF, CHD and stroke). As shown, various components of the R-E score showed different levels of associations with CVD outcomes. Specifically: 1) All of the six components were significantly associated with HF in the demographic adjusted models. However, after further adjustments for CVD risk factors and potential confounders (model 2), QRSAMP and QRSDUR lost their significant associations with HF, and when all the six components were included in the model (model 3), LAXDEV lost its significant association with HF as well; 2) Only LVSTR and LAXDEV were significantly associated with CHD in all models; and 3) Only LVSTR and INTRNS were significantly associated with incident stroke in all models, with QRSAMP only showing significant association in the demographic adjusted model.

TABLE 8
Baseline Romhilt/Estes score components and risk of incident heart failure,
coronary heart disease and stroke

| Score Components | Model | Incident HF | Incident CHD | Incident Stroke |
|---|---|---|---|---|
| QRSAMP | Model 1^{a} | 1.53 (1.12-2.09)^{‡} | 1.13 (0.80-1.59) | 2.20 (1.47-3.27)^{‡} |
| | Model 2^{b} | 1.27 (0.92-1.75) | 1.03 (0.72-1.46) | 1.45 (0.96-2.21) |
| | Model 3^{c} | 1.05 (0.75-1.45) | 0.91 (0.63-1.30) | 1.33 (0.87-2.04) |
| PTFV1 | Model 1^{a} | 1.94 (1.34-2.80)^{‡} | 1.46 (0.98-2.17) | 1.16 (0.58-2.34) |
| | Model 2^{b} | 1.76 (1.20-2.58)^{‡} | 1.39 (0.93-2.08) | 1.09 (0.54-2.21) |
| | Model 3^{c} | 1.75 (1.19-2.57)^{‡} | 1.40 (0.93-2.10) | 1.06 (0.52-2.15) |
| LVSTR | Model 1^{a} | 2.89 (2.36-3.55)^{§} | 2.40 (1.94-2.97)^{§} | 2.22 (1.58-3.11)^{§} |
| | Model 2^{b} | 2.13 (1.72-2.63)^{§} | 1.76 (1.41-2.20)^{§} | 1.54 (1.09-2.18)^{†} |
| | Model 3^{c} | 2.09 (1.68-2.59)^{§} | 1.75 (1.40-2.19)^{§} | 1.48 (1.04-2.11)^{†} |
| LAXDEV | Model 1^{a} | 1.50 (1.23-1.84)^{§} | 1.45 (1.21-1.75)^{§} | 1.12 (0.78-1.59) |
| | Model 2^{b} | 1.24 (1.01-1.53)^{†} | 1.36 (1.13-1.64)^{‡} | 0.99 (0.69-1.42) |
| | Model 3^{c} | 1.21 (0.99-1.49) | 1.33 (1.10-1.60)^{‡} | 0.98 (0.68-1.41) |
| QRSDUR | Model 1^{a} | 1.11 (1.00-1.22)^{†} | 1.08 (0.98-1.18) | 1.02 (0.87-1.19) |
| | Model 2^{b} | 1.04 (0.94-1.15) | 1.08 (0.99-1.18) | 0.99 (0.85-1.15) |
| | Model 3^{c} | 1.01 (0.91-1.12) | 1.07 (0.98-1.18) | 0.95 (0.81-1.12) |
| INTRNS | Model 1^{a} | 1.60 (1.25-2.06)^{‡} | 1.05 (0.82-1.34) | 1.59 (1.08-2.36)^{†} |
| | Model 2^{b} | 1.48 (1.14-1.91)^{‡} | 1.01 (0.79-1.30) | 1.55 (1.05-2.31)^{†} |
| | Model 3^{c} | 1.46 (1.13-1.89)^{‡} | 0.99 (0.76-1.27) | 1.53 (1.03-2.29)^{†} |

^{†}Denotes P < 0.05;
^{‡}P < 0.01;
^{§}P < 0.001 for P values of hazard ratios.
CHD—coronary heart disease;
HF—heart failure
^{a}Model-1: Adjusted for age, sex and race;
^{b}Model-2: Adjusted for variables in model 1 plus field center,, body mass index, systolic blood pressure, smoking status, education, hypertension, diabetes mellitus, family history of coronary heart disease, ratio of total cholesterol/high-density lipoprotein, blood glucose, serum creatinine, and uric acid (all at baseline).
^{c}Model-3: Adjusted for variables in Model 2 plus all of the six R-E score components.

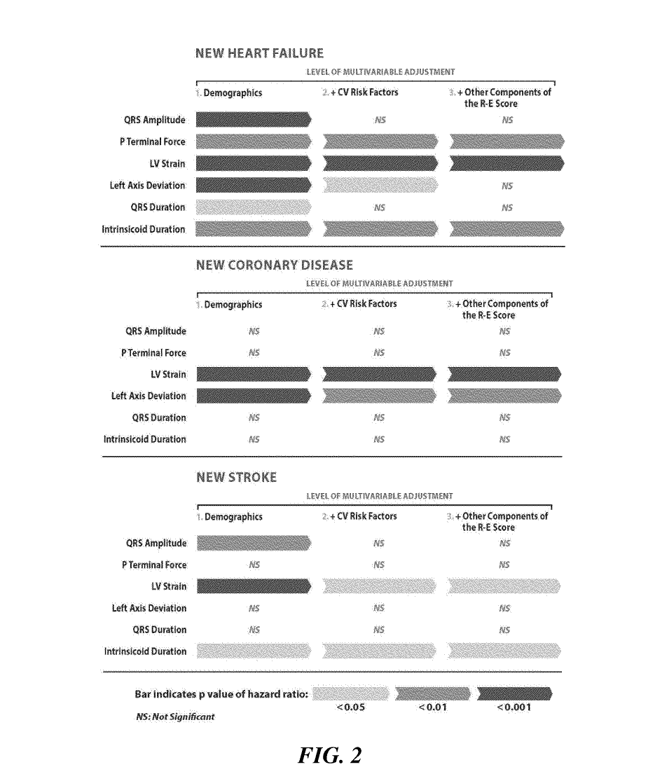

The nature and extent of the differing profiles described above can be better visualized in FIG. 2. This graph illustrates the fact that the six ECG elements of the risk score are all different from each other, and each indicates a different pathophysiological state. If each of these ECG elements were measuring the same thing, it should not matter which CV disease caused the ECG abnormality. All of these six components predict composite heart disease, but when three different “corrections” are applied, they are seen to be different. The first one, QRS amplitude, is a powerful predictor for new heart failure but not at all for new coronary heart disease. The pattern of differences in response to increasing levels of correction enables the prediction of the type of cardiovascular disease.

### Discussion

There were at least two key findings from this analysis. First, a R-E score greater than 4 points (compared to R-E=0 points) was predictive of CVD, CHD, HF and stroke. Second, different components of the R-E score showed different levels of associations with different CVD outcomes, as seen in Table 8 and FIG. 2. Our results showed that the six components of the R-E score were unique in their relationship with different CVD outcomes and may indicate a different predecessor state. Without being bound by theory, it is believed that each of the six ECG findings are a unique electrical biomarker, sharing with the others the ability to predict LVH, all-cause mortality, and incident CVD, but each, individually, predicting a different antecedent pathophysiological state, and a different clinical outcome as well.

Evidence that ECG-LVH and cardiac mass/volume are not directly related comes from a number of independent observations. First is the long recognized fact that many individuals have increased cardiac mass/volume and no ECG findings. Most recognized ECG-LVH diagnosis systems have a sensitivity well below 50% and usually below 30% (18). In 2001, Sundstrom and colleagues (12) reported that Echo-LVH and ECG-LVH predicted mortality independently of each other in a population of elderly Swedish men. Bacharova and colleagues (19) showed that both ECG-LVH and MRI-LVH predicted mortality to the same general level, but differed widely in their detection of LVH, leading to the conclusion that the two methods were likely to be distinct but somehow related phenotypes.

More evidence is found in genetic studies. Mayosi and coworkers (20) found that Sokolow-Lyon voltage measures of LVH displayed a greater heritability than echocardiographic measures of LVH. In a later genome-wide linkage analysis of ECG-LVH and Echo-LVH in families with hypertension, there were stronger linkages for the former, and the genetic determinants of each appeared to be distinct from the other (21). Shah (22) has reported heritability of ECG-LVH identified by four commonly utilized ECG measures, and Hong (23) has reported, in a genome wide association study in a Korean population, variations on the RYR1 gene in patients with ECG/LVH.

The above evidence suggests that ECG wave forms associated with LVH are not rare occurrences with genetic variants. Without being bound by any particular theory, it is believed that a series of genetic variations exist, each of which produces subtle and specific changes in the basic physiology of the myocardial cell. The specific ECG changes might result from the basic genetic defect, or from changes in myocardium initiated by the basic defect acting over many years, such as the accumulation of fibrin within the myocardium, or the development of inflammatory vascular lesions.

### Conclusions

The R-E score is predictive of incident cardiovascular events. The six individual ECG components of the score all share in this predictive ability, but all have an independent and unique ability to predict specific CVD outcomes, defined in this study as HF, CHD, and stroke. The unique nature of response is revealed in the profiles of response of each ECG criterion to multivariable adjustments in the prediction of CV disease, suggesting a different pathophysiological state and outcome.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
1. U.S. Pat. No. 8,346,349, “Method and apparatus for predicting patient outcomes from a physiological, segmentable patient signal.”
2. Romhilt, D W, Estes, E H. A point score system for the ECG diagnosis of left ventricular hypertrophy. Am. Heart J. 1968; 75:752.
3. Sundstrom, J, Lind, L, Arnlov, J, Zethelius, B, Andren, B, and Lithell, H. O. Echocardiographic and electrocardiographic diagnoses of left ventricular hypertrophy predict mortality independently of each other in a population of elderly men. Circulation 2001; 103: 2346.
4. Mayosi, B. M., Avery, P. J., Farrell, M., Keavney, B., and Watkins, H. Genome-wide linkage analysis of electrocardiographic and echocardiographic left ventricular hypertrophy in families with hypertension. Eur. Heart J. 2008; 29:525-30.
5. Estes E H, Zhang Z-M, Li Y, Tereshchenko L G, Soliman S Z. The Romhilt-Estes left ventricular hypertrophy score and its components predict all-cause mortality in the general population. Am. Heart J. 2015; 170 (1):104-109.
6. Okin, P. M., Devereux, R. B., Severer, J. et al. Regression of electrocardiographic left ventricular hypertrophy by Losartin vs. Atenolol. Circulation 2003; 108:684-90.
7. Kurisu, S., Inoue, I., Kawagoe, T, et. al. The decrease in QRS amplitude after aortic valve replacement in patients with aortic valve stenosis. J. Electrocardiol. 2009; 42:410-3.
8. The ARIC investigators. The Atherosclerosis Risk in Communities (ARIC) Study: design and objectives. Am. J. Epidemiol. 1989; 129: 687-702.
9. U.S. Pat. No. 8,615,290, “Seamlessly embedded heart rate monitor.”
10. Moyer, V A, for U.S. Preventive Services Task Force. Screening for coronary heart disease with electrocardiography: U.S. Preventive Services Task Force Recommendation Statement. Ann. Int. Med. 2012; 157:512-19.
11. Bacharova, L, for the Working Group on Electrocardiographic Diagnosis of Left Ventricular Hypertrophy. The first statement of the Working Group on Electrocardiographic Diagnosis of Left Ventricular Hypertrophy. J. Electrocard. 2010; 43:197-9.
12. Sundstrom J, Lind L, Arnlo J, Zethelius B, Andren, B, Lithell H O, et al. Echocardiographic and electrocardiographic diagnosis of left ventricular hypertrophy predict mortality independently of each other in a population of elderly men. Circulation 2001; 103:2346-51.
13. Okin P M, Devereux R B, Liu J E, Oikarinen L, Jern S, Kjeldsen S E et al. Regression of electrocardiographic left ventricular hypertrophy predicts regression of echocardiographic left ventricular mass: the LIFE study. J. Hum. Hypertension 2004; 18:403-9.
14. Rosamond W D, Folsom A R, Chambless L E, Wang C H, McGovern P G, Howard G, Copper L S, Shahar E. Stroke incidence and survival among middle-aged adults: 9-year follow-up of the atherosclerosis risk in communities (aric) cohort. Stroke 1999; 30:736-743.
15. Loehr L R, Rosamond W D, Chang P P, Folsom A R, Chambless L E. Heart failure incidence and survival (from the atherosclerosis risk in communities study). Am. J. Cardiol 2008; 101:1016-1022.
16. Soliman E Z, Lopez F, O'Neal W T, Chen L Y, Bengtson L, Zhang Z M, Loehr L, Cushman M, Alonso A. Atrial fibrillation and risk of st-segment-elevation versus non-st-segment-elevation myocardial infarction: The atherosclerosis risk in communities (aric) study. Circulation. 2015; 131:1843-1850.
17. White A D, Folsom A R, Chambless L E, Sharret A R, Yang K, Conwill D, Higgins M, Williams O D, Tyroler H A. Community surveillance of coronary heart disease in the atherosclerosis risk in communities (aric) study: Methods and initial two years' experience. J. Clin. Epidemiol. 1996; 49:223-233.
18. Levy D, Labib S B, Anderson K M, Christiansen J C, Kannel W B, and Castelli W P. Determinants of sensitivity and specificity of electrocardiographic criteria for left ventricular hypertrophy. Circulation 1990; 81:815-820.
19. Bacharova L, Chen H, Estes E H, Mateasik A, Blumke D A, Lima J A C, Burke G L, and Soliman E Z. Determinants of discrepancies in detection and comparison of the prognostic significance of left ventricular hypertrophy by electrocardiogram and cardiac resonance imaging. Am. J. Cardiol. 2015; 115:515-522.
20. Mayosi B M, Keavney B, Kardos A, Davies C H, Ratliffe P J, and Watkins H. Electrocardiographic measures of left ventricular hypertrophy show greater heritability than echocardiographic left ventricular mass. Eur. Heart J. 2002; 23:1963-1971.
21. Mayosi B M, Avery P J, Farrall M, Keavney B, and Watkins H. Genome-wide linkage analysis of electrocardiographic and echocardiographic left ventricular hypertrophy in families with hypertension. Eur. Heart J. 2008; 29:525-530.
22. Shah S, Nelson C P, Gaunt T R, van der Harst P, Barnes T, et. al. Four genetic loci influencing electrocardiographic indices of left ventricular hypertrophy. Circulation Genetics 2011; 4:626-635.
23. Hong K-W, Shin D-K, Lee S-H, et al. Common variants in RYR1 are associated with left ventricular hypertrophy assessed by electrocardiogram. Eur. Heart J. 2011; 33:1250-1256.

1. A method for predicting risk of adverse cardiovascular events and/or mortality in an individual comprising the steps of:
a. recording an electrocardiogram from the individual;
b. analyzing the electrocardiogram to detect the presence of wave form elements;
c. calculating a risk score based on the presence of the wave form elements; and
d. predicting risk of adverse cardiovascular events and/or mortality based on the risk score.

2. The method of claim 1, wherein calculation of the risk score is based on a longitudinal assessment of wave form elements and adverse cardiovascular events and mortality in a population-based cohort study.

3. The method of claim 2, wherein the longitudinal assessment of wave form elements and adverse cardiovascular events and mortality in a population-based cohort study comprises analysis of the pattern of occurrence, intensity, and statistical response of wave form elements to predict specific adverse cardiovascular events and mortality.

4. The method of claim 3, wherein the pattern of occurrence, intensity, and statistical response of wave form elements are also used to predict the presence of one or more genetic abnormalities in the individual.

5. The method of claim 3, wherein the pattern of occurrence, intensity, and statistical response of wave form elements are also used to predict the presence or absence of one or more biomarkers in the individual.

6. The method of claim 1, wherein the risk score is used to measure the efficacy of a medication, a surgical procedure, a dietary regime, or a treatment program in the individual, wherein an increase in the risk score is indicative of poor efficacy and a reduction in the risk score is indicative of positive efficacy.

7. The method of claim 1, wherein total all-cause risk of mortality is predicted for an individual based on all identified wave form elements, and wherein each wave form element is rated and entered into the total risk score.

8. The method of claim 7, wherein the total risk score is used to measure the efficacy of a medication, a surgical procedure, a dietary regime, or a treatment program in the individual, wherein an increase in the total risk score is indicative of poor efficacy and a reduction in the total risk score is indicative of positive efficacy.

9. The method of claim 1, wherein steps 1(a) to 1(c) are performed by a compatible recording instrument programmed to detect, quantitate, and analyze the wave form elements and calculate the risk score based upon the wave form elements.

10. The method of claim 9, wherein the compatible recording instrument is selected from the group consisting of an ECG recorder and analyzer, a computer, and a portable device.

11. The method of claim 10, wherein the portable device is a hand-held mobile device.
